(19) United States
(12) Patent Application Publication
(10) Pub. No.: US 2026/0306310 A1
(43) Pub. Date: Oct. 8, 2026

(54) Absorbent Undergarment with Two Compartments

(71) Applicant: William Joseph Drasler, Minnetonka, MN (US)

(72) Inventor: William Joseph Drasler, Minnetonka, MN (US)

(21) Appl. No.: 19/453,618

(22) Filed: Jan. 20, 2026

Related U.S. Application Data
(60) Provisional application No. 63/784,361, filed on Apr. 7, 2025.

(51) Int. Cl. A61F 13/495; A61F 13/49; A61F 13/496

(52) U.S. Cl. CPC A61F 13/495; A61F 13/49011; A61F 13/496; A61F 2013/49022; A61F 2013/4953

(57) ABSTRACT

A disposable absorbent undergarment has elastic frontal and rear straps to position, and provide sealing for a separator pad against perineal tissues in the crotch and create two fluid-tight compartments between the anus and urethra blocking fecal matter from contacting the urethra and preventing urinary tract infections. Straps are adjustable to position the separator pad accurately and comfortably. Two seals are formed across the inguinal-gluteal region and around each leg to ensure exudate cannot exit.

### CROSS REFERENCE TO RELATED APPLICATIONS

This patent application makes reference to and thereby incorporates all information found in the provisional patent application No. 63/784,361 entitled Absorbent Undergarment With Two Compartments filed 7 Apr. 2025 by William J. Drasler.

### BACKGROUND

Absorbent undergarments are used by adults with fecal or urinary incontinence to collect and absorb exudate matter that can then be properly disposed. Such absorbent undergarments can also be used to collect and absorb exudate matter from babies that are not yet able to control bowel movements (BM) or urinary outflow. Current absorbent undergarments do not adequately form a fluid-tight seal between the anus and the genitalia along the perineal tissues that would prevent the passage of fecal material into the region of the genitalia and into contact with the urethral opening or genital tissues. Contact of fecal matter with the urethra can lead to detrimental urinary tract infections that can require expensive hospital visits and extensive antibiotic treatments which can lead to sepsis and difficult recovery.

What is needed is a disposable absorbent undergarment that forms a fluid-tight seal with the perineal tissues and prevents the passage of fecal matter into contact with the genitalia and urethra. The undergarment should absorb and contain the bodily exudate matter within its confines until it is properly disposed.

### SUMMARY

The present invention is a disposable absorbent undergarment that is intended to be discarded after a short term of use of approximately one day for most individuals, for example. The undergarment has a separator pad located between the anus and the genitalia and between the two legs of an individual. An unbonded edge or contact surface of the separator pad forms a leak-tight seal with the perineal skin tissues by application of a compressive force of the separator pad against the perineal tissues and compression of the unbonded edge of the separator pad. The separator pad is held with a force against the perineal tissues by a pad support. The pad support is comprised of two elastic front straps, two elastic rear straps, and a belt located along the individual's waist to which the straps are attached. The front straps extend from the front belt at locations along an inside aspect of the leg adjacent to or radially outwards from the inguinal ligament region of the individual; the front straps extend along each inguinal ligament region to the crotch area where they each can attach to opposite ends of the separator pad or the bottom base surface of the separator pad, while leaving the contact surface unattached an free to contact and compress as it makes contact with the perineal tissues. The front straps provide a forward component (toward the urethra) and lateral component (outwards toward the sides of the individual) of the tensile forces of the straps to keep the separator pad on center between the legs and positioned properly with a forward force directed toward the genitalia and urethral opening. The front straps each apply a force toward the inguinal tissue along the inner aspect of the leg that prevents leakage of exudate material between the fluid-impermeable contact of the absorbent garment and the inguinal tissues or inner aspect of the leg. The front straps are directed at an angle laterally outwards relative to the centerline of the undergarment, the centerline extending centrally from the front belt to the rear belt and passing through the separator pad. The rear straps extend from the rear belt along the side of the individual's hip along an outside aspect of the leg and follow along the gluteal tissues of the gluteus maximus muscle to the crotch area where they each can attach to opposite ends of the separator pad along the base surface or end surface of the separator pad ensuring that the contact surface of the separator pad is unattached or unbonded in order to provide direct contact with the perineal tissues and compress to form a seal with the perineal tissues. The rear straps provide a rearward component and lateral component outwards of the tensile force of the straps to keep the separator pad on center between the legs and positioned properly with a rearward force directed toward the anus. The rear straps are directed at an angle laterally outwards relative to the centerline of the undergarment. The rear straps each apply a force toward the gluteal tissues along the outer aspect of the leg that prevents leakage of exudate material between the fluid-impermeable contact of the absorbent garment and the gluteal tissues or outer aspect of the leg. The front straps and rear straps provide a tensile force against the separator pad that transmits through the separator pad thereby compressing the separator pad and thereby applying a compressive force due to compression of the separator pad onto the perineal tissue and making a fluid-tight seal with the perineal tissues.

The front and rear straps on each side of the undergarment can attach to the ends of the separator pad near the leg on that same side; the straps can extend further along the base edge of the separator pad, the base edge being opposite to the unbonded edge that contacts the perineal tissue. The straps collectively exert a tensile force of 1-5 lbs. onto the separator pad as they are stretched from the separator pad to the belt by an amount of 20 percent (range 5-40 percent). The tensile force of the straps cause the separator pad to undergo a compression and generate a compressive force of 1-5 lbs. against the perineal tissue. The belt is an elastic belt with belt openings located on each side of the belt between each front strap and its neighboring rear strap. The belt openings allows the pad support to be placed onto the individual's crotch region in a manner similar to placing a standard disposable diaper onto a baby's bottom.

A jacket comprised of an inner liner, a cover, and an absorbent later contained between the cover and the inner liner can be placed on the garment side of the pad support. The jacket is located between the two rear straps (i.e., from one rear strap to the other rear strap) with attachments to the two rear straps extending from the rear portion of the belt to the separator pad. The jacket is further located between the two front straps with attachments to the two front straps extending from the front portion of the belt to the separator pad. The attachments of the straps to the jacket can be made by extending each strap through a channel or by direct attachment of the jacket to each strap in a manner that allows each strap the ability to stretch under a tensile force. With the straps located on the inside or body side of the jacket, the straps makes a leak-tight seal or contact with the inguinal and gluteal tissues and thereby provides a fluid impermeable contact of the absorbent undergarment with the inguinal tissues (along the inside aspect of the leg) and gluteal tissues (along the outside aspect of the leg). With the straps extending through channels that are attached to the jacket, the jacket makes a leak-tight seal with the inguinal tissues and gluteal tissues and thereby provides a fluid-impermeable contact of the absorbent undergarment with the inguinal and gluteal tissues.

The jacket provides absorbent and containment compartments between the individual's skin in the crotch region and the jacket for collection of fecal matter and urine. The cover provides a fluid-tight layer to the undergarment that does not let exudate matter pass through; the cover can be formed from a thin polymeric material such as Dacron or nylon film, for example. The inner liner is a porous material that is in contact with the skin of the individual and allows fluids to easily pass through to reach the absorbent layer; it can be formed from a fibrous polymeric material or a natural fibrous material formed into a thin film, for example. The absorbent material contained between the cover and inner liner can be polymeric fibrous material that is hydrophilic and able to hold fluids found in the exudate of the body. The jacket can be formed to contain or be positioned adjacent to all or portions of the front and rear straps and can be attached to the straps in a variety of ways, the straps can alternately be configured to move relative to or adjacent to the jacket and in a manner that prevents leakage of fluid between the absorbent undergarment and the inguinal tissues or gluteal tissues.

The jacket can be attached or bonded via a fluid-tight bond to the base edge of the separator pad which is opposite to the unbonded edge or contact surface of the separator pad. The jacket can also form a fluid-impermeable attachment along all or a portion of each end of the separator pad and can form a fluid-impermeable attachment to each of the front and rear straps via a fluid-tight bond, particularly if the jacket is located on the garment side of the straps. The absorbent undergarment forms a fluid-tight contact and seal with the inguinal tissues and gluteal tissues extending along the paths of the straps from the belt to the separator pad. Attachment of the jacket to the separator pad and to the straps of the pad support can provide a formation of two separate compartments that are separated by a fluid-tight seal of the separator pad to the perineal tissues, one for fecal matter, the rear compartment, and one for urine, the front compartment. The only passage for leakage between the rear compartment and the front compartment is between the unbonded edge or contact surface of the separator pad and the perineal tissue to which it makes contact and compresses forming a fluid-tight seal. This perineal leak path is obviated by proper positioning of the separator pad against the perineal tissue and by application of a tensile force by the straps onto the separator pad and this tensile force causing a compression force within the separator pad to be transmitted onto the perineal tissue forming the fluid-tight seal between the front and rear compartment for collection of exudate matter.

The separator pad is formed from a material that has a compression strength and resiliency and can to transmit a force applied to it via the straps toward the perineal tissue to form a fluid-tight seal with the perineal tissue. The separator pad can be formed with two portions, a separator tip that is placed into contact with the perineal tissue and a separator pad base that is sealingly attached to the jacket and attached the straps. The separator pad can alternately be formed from a single contiguous material that comprises both the separator pad base and separator pad tip. The separator pad base can provide a space between the perineal tissue and the jacket into which exudate can be collected and provides a barrier to prevent fecal exudate from access to the urethra which could otherwise potentially lead to urinary tract infection. The separator pad tip can also be rounded from front to back (along the centerline of the absorbent undergarment) forming a small contact area that is able to compress easily against the perineal tissues and for a fluid-tight seal.

The jacket can have a leg rim attached to the jacket extending around each leg of the individual. The garment rim can extend from the front and rear strap on one side of the undergarment laterally outwards from the front strap and rear strap on each side of the undergarment to the upper and lower belt; the leg rim extends for a distance of 1-5 inches from the straps and belt to surround the leg. The leg rim has a construction that can be the same or similar to the jacket and has an elastic band that makes fluid-sealing contact along the perimeter of the leg. The leg rim ensures that any leakage of exudate past the front or rear straps between the individual's skin in the inguinal or gluteal region and the absorbent undergarment and exudate is not allowed to escape out of the undergarment and is contained or absorbed by the leg rim. The undergarment of the present invention therefore has two fluid-tight seals or contacts between the undergarment and the individual's skin. One seal or contact is made by forces of the straps pushing the undergarment into sealing contact with the inguinal and gluteal tissues. The other contact and seal is made between the elastic of the leg rim and each of the legs of the individual.

During initial placement of the absorbent undergarment, positioning of the separator pad at a proper location between the anus an the genitalia is important to ensure that the two compartments are positioned properly between the anus and the genitalia with a fluid-tight seal between them. A positioning handle can be located on the garment outer side (i.e., the outer side of the undergarment opposite to the body side facing the individual's body) of the jacket at a location adjacent to the separator pad. The positioning handle can be further attached through the jacket to the base edge of the separator pad or to the crotch end of the straps. The individual or an attendant can move the undergarment towards the front or rear, and can move the undergarment from side to side to place the separator pad in the proper location between the genitalia and anus per the direction of the individual being fitted with the undergarment using the thin flexible handle. Once the position of the separator pad is initially established, the belt can be secured around the individual's waist via tabs to close the belt openings between the belt front and belt rear. The separator pad can be readjusted if necessary via the handle, moving the belt, or via application of tension to an adjustable strap to properly position the separator pad for comfort to the individual and provide sealing contact with the perineal tissue.

The belt can have a taper such that an upper portion of the belt has a belt upper perimeter or belt upper length that is smaller than a lower portion of the belt having a belt lower perimeter. Placing the tapered belt smaller upper belt perimeter over the hips of the individual can assist in keeping the belt from slipping downwards towards the legs. The belt can also be coated with a porous silicone or polyurethane material to form a grip with the individual's skin and prevent slippage of the undergarment downwards towards the individual's legs. The snug fit of the front straps in the inguinal ligament regions and the rear straps located below the gluteal tissues naturally locates the straps above the wide leg femoral muscle of the leg and prevents the undergarment from moving downwards thereby maintaining a forceful and sealing contact of the separator pad with the perineal tissue.

In one embodiment the front straps and rear straps can be attached to the belt via an adjustable strap cinch. Each strap can be pulled by the individual (i.e., some or all straps can be adjusted individually) to adjust or move the separator pad forward, rearward, or laterally or to obtain a greater (or lesser) force of the separator pad relative to the perineal tissue. A push button spring-activated cinch can be located at each strap-belt attachment to allow for full positioning of the separator pad and ensure a comfortable fit with the individual and effective seal contact of the separator pad with the perineal tissue. With the cinch button pushed, the individual can pull a strap to increase the tensile forces in the strap and thereby pull the separator pad in the lateral and forward/rearward direction of that strap. Release of the cinch button will lock the tensile forces into the strap between the belt and the separator pad. Further pushing of the cinch button will cause the strap to no longer be attached to the belt and the tensile forces of that strap to be reduced or released.

Other materials have been contemplated for the separator pad. In one embodiment, an inflatable balloon can be placed within a foam structure of a separator pad, for example. The inflatable balloon can be inflated via a bulb located near the belt. The inflatable bulb can be squeezed via hand action to cause the separator pad to make increased forceful contact and seal with the perineal tissue. Also, the balloon can be inflated further to maintain sealing contact of the separator pad with the perineal tissue due to potential slippage of the undergarment downwards towards the legs. A release valve can be activated by the individual to reduce the compressive force of the separator pad against the perineal tissues.

A positioning member can be attached or contiguous with the separator pad to assist in positioning the separator pad between the anus and the genitalia. A front positioning member can extend from the separator pad toward the genitalia and extend into the vaginal opening of a female individual. A rear positioning member can extend from the separator pad toward the anus and extend into the anus of any individual user of the absorbent undergarment. The positioning member is formed from a flexible polymeric material to provide comfort to the individual while maintaining adequate structural strength to maintain the separator pad into position against the perineal tissues between the anus and the genitalia.

### BRIEF DESCRIPTION OF THE DRAWINGS

FIG. 1 is a perspective side view of the separator pad of an undergarment and the separator pad support structures including the front straps, rear straps, belt front, and belt rear.

FIG. 2 is a perspective frontal view of the separator pad of an undergarment and the separator pad support structures including the front straps, rear straps, belt front, and belt rear.

FIG. 3A is a perspective side view of the separator pad and the separator pad support structures of an undergarment superimposed onto a cross-sectional view of an individual from the side through the crotch region with the separator pad in contact with perineal tissues and in contact with the centerline of the undergarment jacket.

FIG. 3B is a cross-sectional view of the jacket of the undergarment with a liner on the body side, a cover on the outer garment side, and an absorbent layer in between the liner and the cover.

FIG. 4 is a perspective frontal view of the separator pad and the separator pad support structures of an undergarment superimposed onto a cross-sectional view of an individual from the front through the crotch region with the separator pad in contact with perineal tissues and in contact with the undergarment front jacket.

FIG. 5 is a perspective rear view of the separator pad and the separator pad support structures of an undergarment superimposed onto a cross-sectional view of an individual from the rear through the crotch region with the separator pad in contact with perineal tissues and in contact with the undergarment rear jacket.

FIG. 6 is a top view of the undergarment spayed out flat and viewed from the body side showing a tapered belt front, tapered belt rear, front straps, and rear straps supporting the separator pad to prevent front-to-rear movement and lateral movement of the separator pad.

FIG. 7 is a close-up plan frontal view of the separator pad and the attachment of the front straps, rear straps, and jacket to the separator pad and showing a positioning handle attached to the jacket, support structure, and the separator pad.

FIG. 8 is a close-up plan side view of the separator pad and the attachment of the front straps, rear straps, and jacket to the separator pad.

FIG. 9A shows a semi-perspective frontal view of an adjustable front strap or rear strap extending from a cinch located on the belt, through a channel located on the outside or within the jacket, and attached to the separator pad to provide positioning of the separator pad from front to back or laterally.

FIG. 9B shows a cross-sectional view of a strap channel for a front strap or rear strap formed from a channel cover located on the garment side of the jacket cover.

FIG. 10 is a perspective frontal view of the separator pad support structures for the undergarment having an inflatable balloon as part or all of the separator pad structure and having an inflation bulb located on or near the belt for the individual to inflate the balloon via a tube connecting the bulb with the balloon.

FIG. 11 is a side view through the crotch region of an individual with the separator pad in contact with the perineal tissues; the separator pad has positioning members attached to the separator pad to provide front-to-rear positioning of the separator pad; a front positioning member can extend into a vaginal opening, a rear positioning member can extend into the anus.

### DETAILED DESCRIPTION

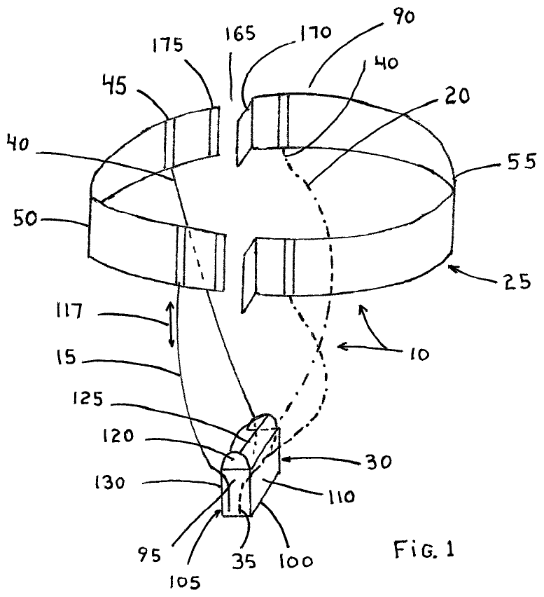

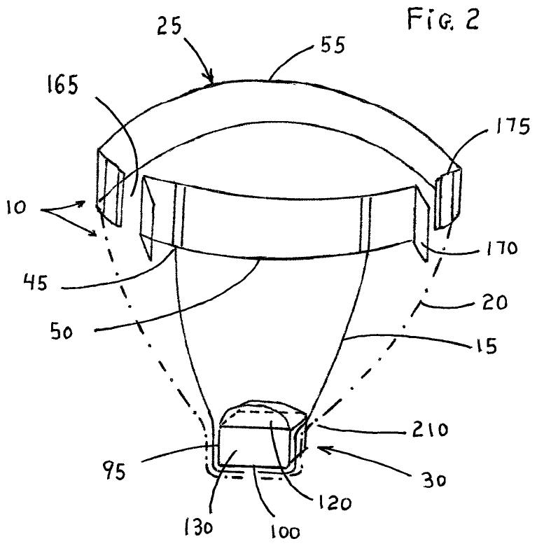

FIGS. 1, 2, 3A, 4, 5, and 6 show perspective side views, front views, and a rear view of an undergarment 5, and show a pad support 10 that is a component of an undergarment 5 of the present invention. The pad support 10 is comprised of two front straps 15, two rear straps 20, and a waistband or belt 25. A separator pad 30 is attached to the crotch ends 35 of front straps 15 and rear straps 20 of the pad support 10; the belt ends 40 of the straps 15, 20 are attached to the belt 25 via belt-strap attachments 45. The pad support 10 is intended to support the separator pad 30 by applying a tensile force 117 via elastic stretch of the straps 15, 20 onto the separator pad 30 in a direction upward towards the belt 25, forwards towards the belt front 50, and rearwards toward the belt rear 55 to keep the separator pad 30 located against the perineal tissue 60 between the anus 65 and the genitalia 70 and between the legs 75 of the individual. The front straps 15 and rear straps 20 are directed at an angle with a lateral componency (i.e., to the sides) relative to the centerline 80 of the undergarment 5 (see FIG. 6) such that each strap directs an outward force 85 laterally toward the side of the undergarment 5 and away from the garment centerline 80. This lateral outward force 85 keeps the separator pad 30 centered relative to the perineal tissues 60 between the individual's legs 75. The crotch ends 35 of the front straps 15 and rear straps 20 on one belt side 90 can be attached to one end of the separator pad 30 and can further extend and attach to the separator pad 30 along the separator pad base edge 100 or pad base bottom surface 100 of the separator pad 30 or separator pad front surface 105 or separator pad rear surface 110. Similarly, the front strap and rear strap on the opposite belt side 90 can be attached to the opposite end of the separator pad 30 and along the separator pad base edge 100 or separator pad front surface 105 or separator pad rear surface.

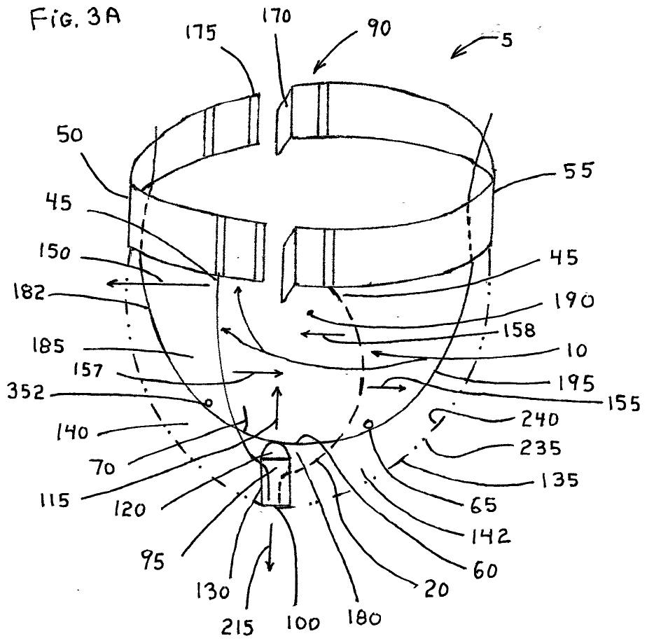

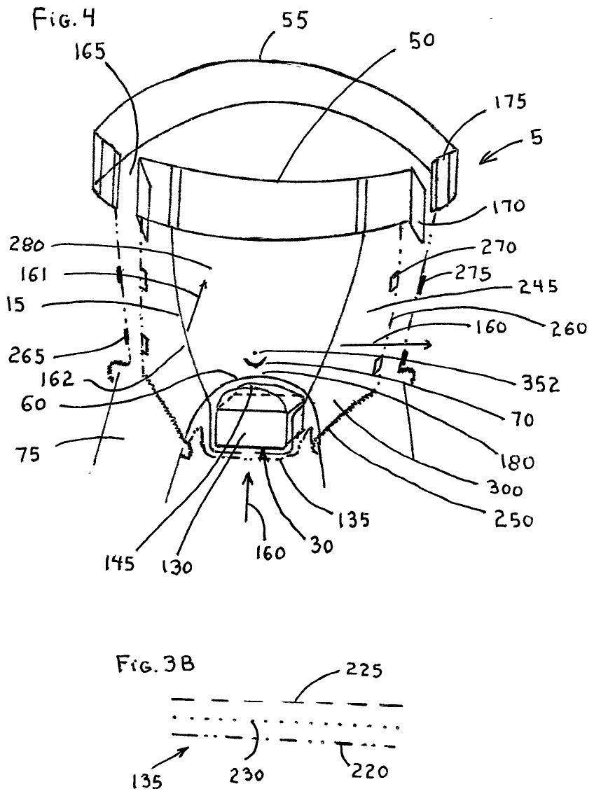

The separator pad 30 can be formed from a polymeric foam that has a compression strength and resiliency (i.e., ability for elastic return to its initial shape following compression and removal of the compression) to transmit a compressive force 115 applied to it via the tensile force 117 from the straps 15, 20 toward the perineal tissue 60 to form a fluid-tight seal with the perineal tissue 60. The polymeric foam can be a closed cell foam to prevent fluid transmission through the foam separator pad 30. The separator pad 30 can be formed with two portions, a separator pad tip 120 having an unbonded edge 125 or tissue contact surface 125 that is placed into contact with the perineal tissue 60 and a separator pad base 130 that is fluid-sealingly attached to a jacket 135 (see FIGS. 3A and 3B).

The separator pad base 130 can provide a front compartment 140 and rear compartment 142 between the perineal tissue 60 and the jacket 135 into which urine or fecal matter can be collected. The separator pad tip 120 can be rounded from one pad end 95 nearest one leg to the other pad end 95 nearest the other leg with a pad tip peak 145 along the centerline 80 (see FIGS. 4 and 6) of the undergarment 5. The separator pad tip 120 can also be rounded from front to back forming a bullet-shaped profile having a pad tip center (see FIG. 3A) with a separator pad tip thickness 350 from front to back of only ¼ inch (range 1/16 to ½ inch, for example). The bullet-shape profile will let the unbonded edge 125 or contact surface 125 of the separator pad tip 120 to contact and compress easily against the perineal tissues 60 to form a fluid-tight seal without discomfort to the user. Such polymeric foam for the separator pad 30 can be formed from polyurethane or other polymers or natural materials having compression character and resiliency. The foam applies a compressive force 115 of 1-5 lbs. against the perineal tissues 60 to form the fluid-tight seal; this compressive force 115 is generated by stretching the front straps 15 and rear straps 20 as the undergarment 5 is placed onto the individual.

The separator pad 30 as discussed above is formed from a resilient material that is able to compress when exposed to an externally applied force and return to its naturals state when the force has been removed. The separator pad 30 should be able to be compressed by a force of two pounds or less and undergo a deformational strain of at least 10% in order to form a leak-tight seal with the perineal tissues 50 and maintain a comfortable low force upon the perineal tissues 60. The externally applied force is provided to the separator pad 30 by tensile forces 117 generated due to the tensile forces 117 within the straps 15, 20 due to stretch that is maintained in the elastic front straps 15 and rear straps 20, the tensile force 117 of the straps 15, 20 being transferred to the separator pad 30. Such resilient properties of the separator pad 30 are required in order to generate a compressive force 115 of the separator pad 30 against the perineal tissue 60 and maintain a seal of the separator pad 30 against the perineal tissue 60.

Materials for the separator pad 30 include elastomeric materials including thermoelastic and thermoset polymeric materials. Such materials include polyurethane, silicone, rubber materials, elastic polymers, synthetic elastomers, natural elastic materials, and other copolymers or combinations of elastomeric materials. The separator pad 30 can be formed from a foam that is formed from an elastomeric material such as polyurethane foam, for example. The foam material can be closed cell to prevent passage of fluid exudate material from crossing through the foam separator pad 30. Alternately, the foam can be open cell foam to help absorb fluid exudate and prevent passage of solid exudate from passage across the foam pad from a rear compartment 142 to a front compartment 140, for example. The foam can be a combination of open cell and closed cell foam with the open cell foam facing both the rear compartment 142 and front compartment 140 (for collection of fecal and urine exudate) with closed cell foam in between two layers of open cell foam, for example. The separator pad 30 can also be formed from fibers formed from an elastomeric material that can be thermally or chemically bonded or formed into a separator pad 30 that has resilient character as described.

The front straps 15 and rear straps 20 are formed from an elastic material that is able to generate a collective upwards compressive force 115 of 1-5 lbs. towards the belt 25 of the separator pad 30 onto the perineal tissue 60 as the straps 15, 20 undergo a stretch of 5-40 percent during placement of the undergarment 5 onto the individual. The upward force of the straps 15, 20 pushes the separator pad 30 toward the perineal tissue 60 causing the separator pad 30 to compress against the perineal tissue 60 and form a fluid-tight seal of the unbonded edge 125 or contact surface 125 of the separator pad 30 with perineal tissues 60 of the body as will be discussed in more detail in subsequent figures. The front straps 15 exert a forward force 150 towards the belt front 50, the rear straps 20 exert a rearward force 155 towards the belt rear 55, and the front and rear straps 20 on one side of the belt 25 exert a lateral force 160 onto the separator pad 30 toward that side of the belt 25. The forward force 150 and rearward force 155 generated by the straps 15, 20 are in balance to keep the separator pad 30 from moving either forward or rearward. The lateral forces 160 from the straps 15, 20 on each side of the pad support 10 are in balance to prevent the separator pad 30 from moving from one side to another.

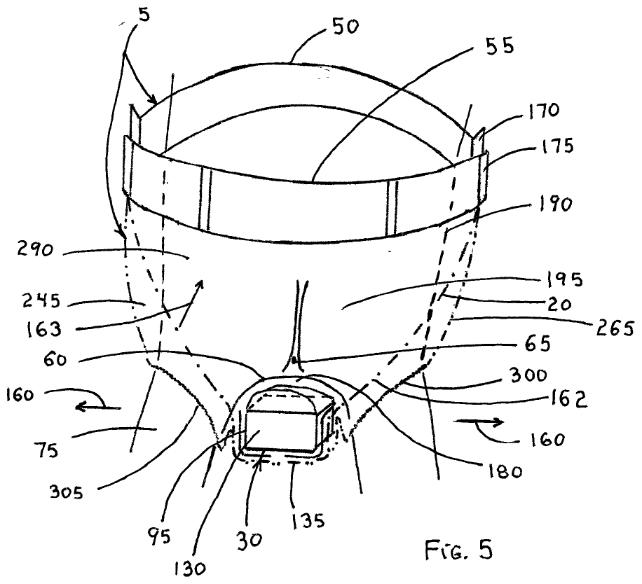

As shown in FIG. 4, the front straps 15 apply a front strap normal force 161 (i.e., perpendicular to the inguinal tissues 185) from the belt 25 to the separator pad 30 in a rearward direction 157 (see FIGS. 3A and 4) against the inguinal tissues 185 to place the absorbent undergarment into a fluid-impermeable contact 162 or fluid-impermeable seal 162 with the inguinal tissues 185 to prevent escape of exudate material across the fluid-impermeable contact 162 between the absorbent garment 5 and the inguinal tissues 185. As shown in FIG. 5, the rear straps 20 apply a rear strap normal force 163 (i.e., perpendicular to the gluteal tissues 195) from the belt 25 to the separator pad 30 in a forward direction 158 (see FIGS. 3A and 5) against the gluteal tissues 195 to place the absorbent undergarment into a fluid-impermeable contact 162 or fluid-impermeable seal 162 with the gluteal tissues 195 to prevent escape of exudate material across the fluid-impermeable contact 162 between the absorbent garment 5 and the gluteal tissues.

The belt 25 has belt openings 165 on each side that allow the undergarment 5 along with its pad support 10 to be placed easily into position in the crotch region 180 of the individual in a manner similar to placing a disposable diaper onto a crotch region 180 of a baby between the baby's legs. The belt openings 165 can be closed using belt tabs 170 that attach to a belt receptacle 175; the belt tabs 170 and belt receptacle 175 can be formed from Velcro, adhesive material, snaps, or other attachment members.

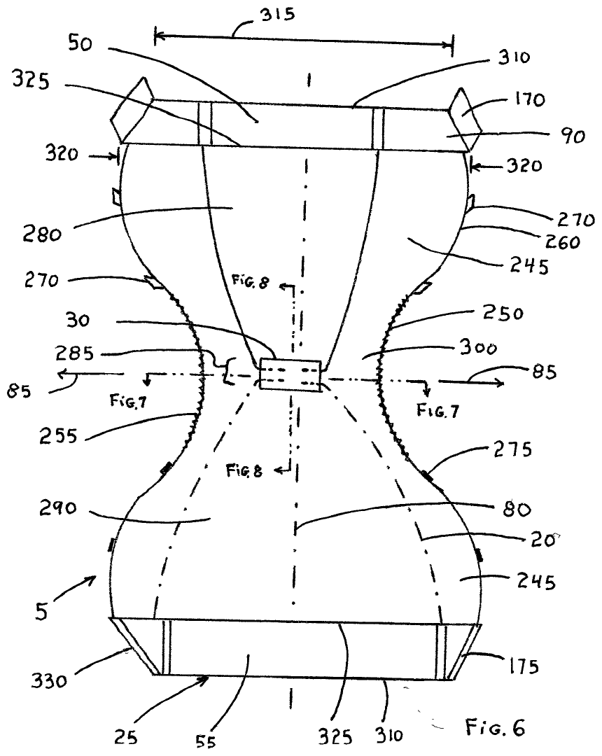

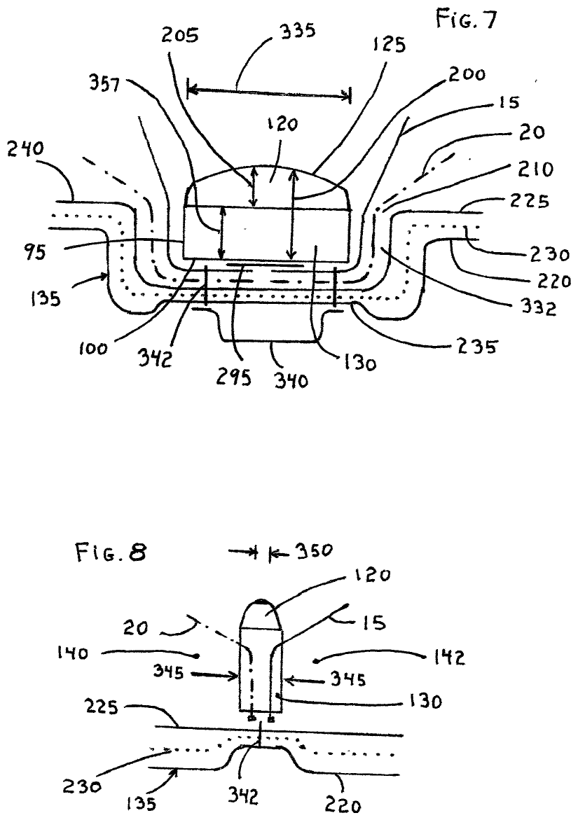

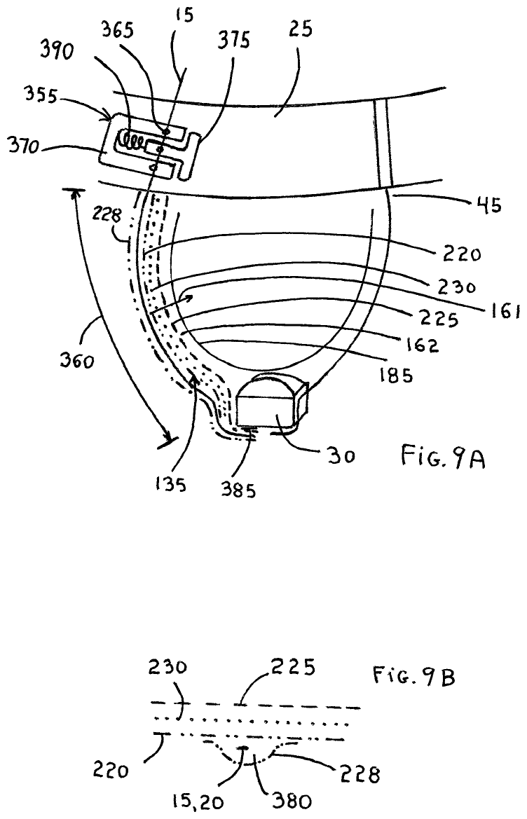

FIGS. 3A, 4, and 5 show a semi-perspective side view, front view, and rear view, respectively, of the disposable absorbent undergarment 5 of the present invention placed onto anatomic structures of an individual. FIG. 3A shows a side view along the centerline 80 of the undergarment 5 and a side view passing through the crotch region 180 of the individual. The front strap extends from the belt-strap attachment at the belt front 50 on the belt side 90 closest to the observer over the abdomen 182 and across the inguinal region 185 to reach one pad end 95 of the separator pad 30 and can attach to the pad base on the separator pad 30. The rear strap extends from a belt-strap attachment at the belt rear 55 on the belt side 90 closest to the observer along the side of the hip 190 and around the gluteal tissue 195 in the gluteus maximus 195 muscle tissue to reach the same pad end 95 of the separator pad 30. The front straps 15 and rear straps 20 on the opposite side can similarly extend and attach to the opposite end of the separator pad 30. The separator pad 30 can have a pad height 200 of 1 inch (range ½-2 inches) (see FIG. 7). The separator pad 30 has a separator pad tip 120 that can be rounded from front to back to more easily deform when making sealing contact with the perineal tissue 60; the separator pad tip 120 is not bonded to the straps 15, 20 or other members such that it is free to compress against the perineal tissue 60 and form a leak-tight seal. The separator pad tip height 205 is ¾ inch (range ¼ inches to 1 inch (see FIG. 7).

The front straps 15 and rear straps 20 on each side of the undergarment 5 provide a compressive force 115 that pulls the separator pad tip 120 into sealing contact with the perineal tissues 60 as seen in FIG. 3A, and hold the separator pad 30 from front to back with a forward force 150 and rearward force 155 that is balanced to keep the separator pad tip 120 located between the genitalia 70 and anus 65. Seen in FIG. 4 is a frontal view of the undergarment 5 placed onto the individual along the crotch region 180 between the two legs 75; the front straps 15 attached to the separator pad 30 hold the separator pad 30 centrally between each leg via lateral forces 160 (toward the side of the individual) and also provide and upward compressive force 115 to hold the separator pad 30 into sealing contact with the perineal tissue 60.

As seen in FIG. 5 from a rear view of the undergarment 5 as viewed along a region between the two legs 75, the rear straps 20 attached to the separator pad 30 also hold the separator pad 30 centrally between each leg via lateral forces 160 and also provide an upward compressive force 115 to hold the separator pad 30 into sealing contact with the perineal tissue 60. The front straps 15 which can connect to the rear straps 20 as they contact (or via their direct individual contact with) the separator pad 30 at the front-rear strap junction 210 (see FIG. 7) follow a pathway from the belt 25 across the inguinal region 185 near the top of the leg and follow an inside aspect of the leg to attach to the separator pad 30. The rear strap follows a pathway from the belt 25 across the outside aspect of the leg and around gluteal tissues 195 to the bottom of the gluteus maximus 195 tissue to attach to the separator pad 30. The snug fit of the front straps against the inguinal tissues 185 in the inguinal region 185 along with the snug fit of the rear strap against the gluteal tissues 195, and both straps 15, 20 residing above the large femoral muscles of the leg will prevent leakage of exudate material between the absorbent undergarment 5 and the inguinal 185 or gluteal 195 tissues of the individual and will prevent the movement in a downward direction 215 (i.e., toward the individual's feet) of the undergarment 5 and will thereby maintain the tensile force 117 provided by the straps 15, 20 onto the separator pad 30 to form a compression force of the separator pad 30 onto the perineal tissue 60 forming a fluid-tight seal and form two separate fluid-tight compartments 140, 142 for collection and absorbance of exudate matter in front of and to the rear of the separator pad 30.

As seen in FIGS. 3A, 3B, 4 and 5, a jacket 135 comprised of an outer cover 220 (or jacket cover 220), an inner liner 225, and an absorbent layer 230 extends from sealing attachment to the belt front 50, extending along with the jacket cover 220 and can have a fluid-tight attachment to the front straps 15 with the jacket 135 located on the garment side of the straps 15, 20. The jacket 135 further extends around the crotch region 180 and has a fluid-tight attachment to the separator pad base edge 100. The jacket 135 can be sealingly attached to the rear straps 20 and sealingly attached to the belt rear 55. Fluid is not able to leak between the straps 15, 20 and the jacket cover 220 which is located on the garment side 235 or outer side of the undergarment 5. The jacket 135 is intended to provide the outer member for two compartments, a front compartment 140 on the front of the separator pad 30 for collection and absorbance of urine and a rear compartment 142 on the rear of the separator pad 30 for collection of fecal matter.

The jacket cover 220 on the garment side 235 is impermeable to fluid to prevent passage of exudate material through the jacket 135. The liner on the body side 240 provides a contact surface with the skin of the individual and allows easy transfer of fluids therethrough to reach the absorbent layer 230. The absorbent layer 230 is able to absorb significant amounts of fluid from the exudate. Other layers can be included within the jacket 135 without deviating from the present invention.

Materials for the jacket cover 220 include polymeric materials that can be formed into a pliable thin film or web that does not allow passage of fluids therethrough. The polymeric film can preferably be thermally bonded or adhesively bonded to other layers of the jacket 135. Polymer currently used in diapers and adult incontinence article jacket cover 220 materials that are suitable for the jacket cover 220 include polypropylene, polyethylene, nylon, polyolefins, and other thermoplastic polymers used in the diaper industry. Materials for the jacket 135 absorbent layer 230 include absorbent fibers formed from synthetic or polymeric materials or from natural materials including cellulose based materials. Polymeric materials for the absorbent materials include polyester, nylon, and other hydrophilic fibers that are able to absorb significant amounts of water based fluids. Other polymeric materials for the absorbent layer 230 include fibers formed from polyolefins that have been treated to make them hydrophilic. The jacket liner can be formed from natural or synthetic fibers including polyester, polypropylene, nylon, polyolefins, and other materials that can be formed into a hydrophilic layer that allows passage of water-based fluid therethrough. The liner can also be formed from a polymeric porous film that is hydrophilic.

The elastic front straps 15 and rear straps 20 make a forceful fit against the inguinal region 185 and gluteal tissues 195 of the body, respectively, to ensure that leakage cannot occur between the inguinal tissues 185 and the undergarment 5. The front straps 15 extend from the belt 25 to the crotch region 180 generating a force against tissues of the inguinal region 185 and preventing leakage of exudate material between the undergarment 5, which which can have the jacket 135 attached to the front straps 15 (when the jacket is on the garment side of the front straps 15) and the tissues of the individual's inguinal region 185. The rear straps 20 extend from the belt 25 to the crotch region 180 making forceful contact along the hip region 190 and gluteal region 195 of the individual and preventing leakage of exudate material between the absorbent undergarment 5 which can be sealingly attached to the rear straps 20 (when the jacket is on the garment side of the rear straps 20) and the individual's gluteal tissues 195.

The elastic straps 15, 20 can be formed from elastic materials that include thermoplastic elastomers, silicone rubber, polyurethane, natural rubber including latex rubber, and other natural and synthetic based elastic materials. Elastic fibers can be formed into the elastic straps 15, 20 of the present invention. The front straps 15 and rear straps 20 can be formed woven elastic fibers and can be formed by enclosing an elastic ribbon into a woven outer sheath, or weaving elastic fibers along with polymeric fibers, for example.

A garment rim 245 which includes a leg rim 300 (see FIG. 6) can be formed from a material similar to the jacket 135 and can extend from the front strap on one side of the undergarment 5 to the rear strap on the same side of the garment in an outward direction away from the centerline 80 of the undergarment 5. The garment rim 245 can be contiguous with all or a portion of the jacket 135. The leg rim 300 on one side (the garment rim 245 on both sides of the undergarment 5 are constructed similarly) of the undergarment 5 has an elastic band 250 at the leg rim edge 255 located away from the straps 15, 20 outwardly away from the centerline 80 of the undergarment 5 in the portion of the garment rim 245 that surrounds the leg on that side of the garment. The garment rim 245 ensures that any exudate material that escapes from the snug fit of the straps 15, 20 and jacket 135 with the inguinal region 185 tissue and gluteus maximus 195 tissue cannot escape from the undergarment 5. An edge tab 270 and edge receptacle 275 hold the front rim edge 260 and the rear rim edge 265 together. The edge tab 270 and edge receptacle 275 can be formed from Velcro, adhesive, various snap devices, or other methods. The present absorbent undergarment 5 has two structures to prevent leakage of exudate out from being contained within the undergarment 5 and exit said absorbent undergarment 5. The garment rim 245 with its elastic band 250 provides one mode of preventing exudate from exiting said undergarment 5. Additionally, the front straps 15 and rear straps 20 provide a force against the inguinal tissues 185 and gluteal tissues 195, respectively, to create an impermeable contact of the undergarment 5 with the inguinal 185 and gluteal 195 tissues that prevents exudate material from exiting said undergarment 5.

FIG. 6 is a top view of the spayed out flat undergarment 5 of the present invention viewed from the body side 240. The front jacket 280 is attached to and extends from the belt front 50 and is attached to and extends between (i.e., from one front strap 15 to another front strap 15) the front straps 15 to the separator pad region 285. At the separator pad region 285, the rear jacket 290 extends contiguously with the front jacket 280. The separator pad 30 sealingly bonded to the jacket 135 at the jacket-pad attachment 295 (see FIG. 7) such that fluid is unable to leak between the jacket cover 220 and the separator pad 30. The rear jacket 290 extends from the separator pad region 285 toward the belt rear 55; the rear jacket 290 is attached to and extends between the rear straps 20 extending to the belt rear 55 where the rear jacket 290 is attached to the belt rear 55.

A garment rim 245 extends laterally outwards from the garment centerline 80, from the front straps 15, and from the rear straps 20 for 1-5 inches; the garment rim 245 can be formed from the same material as the jacket 135 or a modified version of the jacket 135 material with more layers or fewer layers of material. The garment rim 245 includes an impermeable jacket cover 220 to prevent leakage through the garment rim 245. The leg opening region 305 or leg rim 300 of the garment rim 245 has an elastic band 250 attached along the leg rim edge 255 of the garment rim 245 to provide a seal of the garment rim 245 with the leg and tissues extending outside of the undergarment 5. The garment rim 245 can be contiguous with the jacket 135. An edge tab 270 and edge receptacle 275 can be located on various regions of the garment rim 245 to hold the front rim edges 260 of the garment rim 245 to the rear rim edges 265 on each side of the undergarment 5 a various locations including, at the belt 25, between the belt 25 and the leg opening 305, and near the leg opening 305 into proximity with each other.

The belt 25 can have a tapered shape such that the belt upper portion 310 has a belt upper portion length 315 of the belt front 50 and belt rear 55. The belt upper portion length 315 is shorter than the belt lower portion length 320 of the belt lower portion 325. The belt perimeter (i.e., the belt upper portion length 315 for the belt front 50 plus the belt rear 55) for the belt upper portion 310 can be shorter by 1-4 inches than the belt lower portion 325. The tapered belt 330 is intended to fit over the hip region 190 of an individual to help ensure that the undergarment 5 cannot slip downwards; hence the tapered belt 330 ensures that the straps 15, 20 provide an continuous tensile force 117 onto the separator pad 30 and the separator pad 30 under compression provides a fluid-tight seal with the perineal tissue 60. The belt 25 can be coated with a material such as a porous polyurethane or porous silicone, including fibrous polymer materials to provide surface that is temporarily tacky to the individuals skin without an adhesive and allowing for good air flow to the tissues of the waist. The coated belt 25 further ensures that the undergarment 5 cannot slip downwards and thereby maintains a continuous force of the separator pad 30 against the perineal tissue 60.

FIG. 7 shows a close up view of a cross-section of the separator pad region 285 of FIG. 6. The jacket 135 is attached to the separator pad 30 forming a fluid-tight jacket-pad attachment 295. Also seen are the front straps 15 and rear straps 20 extending along and attached to the separator pad ends 95; the front straps 15 and rear straps 20 can also extend into contact and can be attached to the separator pad base edge 100 (that is opposite to the separator pad unbonded edge 125). The straps 15, 20 can also attach to the front 105, rear 110, or end 95 surfaces of the separator pad 30 leaving the unbonded contact surface 125 unattached to provide direct contact with the perineal tissues. The separator pad tip 120 can have a curved shape and can extend away (i.e., toward the perineal tissue 60) from the separator pad base edge 100 without any attachment to other members of the undergarment 5 along its sides or ends thereby allowing the separator pad tip 120 to be easily deformed as it forms a seal against the perineal tissue 60.

The jacket 135 can make a fluid-tight end seal 332 with the pad ends 95 of the separator pad 30 from the separator pad base edge 100 to front-rear strap junction 210 of the rear strap with the front strap on each end of the separator pad 30. Hence fluid leakage cannot occur from a rear compartment 142 to a front compartment 140 across the separator pad 30 as long as a fluid-tight seal is made from the separator pad tip 120 to the perineal tissue 60. The separator pad 30 can have a separator pad height 200 of ½-2 inches, the separator pad tip height 205 can range from ¼-1 inch. The separator pad width 335 can range from 2-5 inches. A soft, flexible, positioning handle 340 is located adjacent to the separator pad 30 on the outer surface or outer side 235 of the jacket 135 and attached to the separator pad 30 or to the front straps 15 and rear straps 20. The positioning handle 340 can be formed from a flexible polymeric cord or ribbon, for example. Movement of the positioning handle 340 by the individual or attendant will result in positioning the separator pad 30 to a location that is comfortable and properly located between the anus 65 and the genitalia 70. A bond 342 can be formed via thermal bonding, adhesive bonding, or other methods to hold the positioning handle 340 to the jacket 135, to hold the jacket 135 to the separator pad 30, and to hold the straps 15, 20 to the separator pad 30.

FIG. 8 is a cross-sectional side view of the separator pad region 285 as shown in FIG. 6. The separator pad 30 can have a pad thickness 345 near the separator pad base 130 of ¾ inch (range ⅜-1.25 inches. The separator pad tip 120 can have a tapered or rounded shape with a separator pad tip thickness 345 of ¼ inch (range 1/16-½ inch. The thin separator pad tip thickness 345 allows for increased ease for positioning the separator pad 30 between the genitalia 70 and the anus 65 without interfering with exudate passage from the urethra 352 or anus 65. The front strap and rear strap can be bonded to each end of the separator pad base 130 below the separator pad tip 120 such that the separator pad tip 120 is free to compress and form a fluid-tight seal with the perineal tissue 60. Fecal matter is thereby unable to pass from the anus 65 across the separator pad 30 into contact with the urethra 352 and cause a urinary tract infection that can be detrimental to the individual and can be costly to treat.

The separator pad base 130 which extends from the front-rear strap junction 210 to the pad base provides a space for collection of fecal matter and body exudate. The jacket 135 is fluid-sealingly attached to the separator pad base 130. The separator pad base height 357 can be 1 inch (range zero to 2 inches) to provide adequate space for exudate collection. Alternately, the base height 357 can be eliminated and the separator pad can be formed from a single material that forms a fluid-tight seal with the perineal tissues and also has a fluid-tight bond with the jacket. Two compartments can be formed in the jacket 135 on each side of the separator pad 30; the compartments can be formed by creating a protrusion or space in the jacket 135 for exudate collection between the individual's skin near the crotch region 180 and the jacket 135.

As seen in FIGS. 7 and 8, the jacket 135 sealingly extends along the separator pad base edge 100 and also extends along the pad end 95 up to the front-rear strap junction 210 of the front and rear straps 20. This fluid-tight seal of the jacket 135 to the separator pad 30 prevents fecal exudate material from crossing the separator pad 30 from the rear compartment 142 on the anal side of the separator pad 30 to the front compartment 140 on the urethral side of the separator pad 30. As seen in FIG. 8, two separate compartments are formed by the separator pad 30, the front compartment 140 and the rear compartment 142. Exudate from the rear compartment 142 is unable to cross the separator pad 30 and enter the front compartment 140.

The front and rear straps 15, 20 of the present invention can be adjustable in length and in tensile force. As shown in FIGS. 9A and 9B, spring-activated cinches 355 can be attached to the belt 25 at belt-strap attachment sites 45 45. The belt-strap attachment sites 45 can be adjacent to the inguinal region 185 on the belt front 50 and adjacent to the hip region 190 on the belt rear 55. The cinches 355 can be used to adjust the strap length 360 and tensile forces of each of the straps 15, 20 individually. Each strap (i.e., each front strap and each rear strap) can be extended through aligned cinch holes 365 in the cinch base and in the cinch button 375. The straps 15, 20 can then extend through an enclosed strap channel 380 formed with a channel cover 228, for example, located on the outside of the jacket 135 extending from the cinch 355 to a strap-pad attachment 385 of the strap to the separator pad 30 in the crotch region 180 of the undergarment 5.

The enclosed strap channel 380 is shown in cross-section in FIG. 9B; the enclosed strap channel 380 can be a continuous channel formed from flexible channel cover 228 material that has a lumen of similar (although slightly larger) size and shape as one of the straps 15, 20. Upon pushing a cinch button 375 on the cinch 355, cinch holes 365 of the cinch base 370 are aligned with cinch holes 365 on the cinch button 375 and the strap can be easily advanced under tension to place that strap under a greater amount of stretch and generating a greater compressive force 115 onto the separator pad 30 by that strap. Upon release of the cinch button 375, a spring 390 causes the cinch holes 365 to no longer align and the strap is held under a stretched condition. Each strap can be individually adjusted to position the separator pad 30; the separator pad 30 can be adjusted forward toward the urethra by tightening the two front straps 15 or rearward toward the anus 65 by tightening the two rear straps 20, for example. The separator pad 30 can be moved laterally to one side by tightening the front strap and rear strap located on that one side. The amount of compressive force 115 exerted by the separator pad 30 onto the perineal tissue 60 can also be controlled by tightening or loosening some or all of the straps 15, 20.

Adjustable straps 15, 20 (ie., either front strap 15 or rear strap 20) can be contained within a separate strap channel 380 as shown in FIG. 9B that is formed by a channel cover 228 located on the garment side 235 of the jacket cover 220. The adjustable straps 15, 20 can alternately be located in separate channels that are attached to the jacket 135 and directs the straps 15, 20 from the belt 25 to the separator pad 30; alternately the adjustable straps 15, 20 can be located within a separate channel contained within the jacket 135. The straps 15, 20 of the present invention as described in all earlier embodiments of this specification can similarly be located in separate strap channels 380; the strap channels 380 can be attached to the jacket 135 or contained within the jacket 135 extending from the belt 25 to the separator pad 30 as described earlier in this specification. In each embodiment of the present invention the front straps 15 place a force against the inguinal tissues 185 and the rear straps 20 place a force against the gluteal tissues 195 to prevent leakage of exudate material between the undergarment 5 contact or seal with the inguinal 185 or gluteal 195 tissues and exiting said undergarment 5.

As shown in FIG. 9A, the front straps 15 apply a front strap normal force 161 (i.e., perpendicular to the inguinal tissues 185) from the belt 25 to the separator pad 30 in a rearward direction 157 (see FIG. 3A) against the inguinal tissues 185 to place the absorbent undergarment into a fluid-impermeable contact 162 or fluid-impermeable seal 162 with the inguinal tissues 185 to prevent escape of exudate material across the fluid-impermeable contact 162 between the absorbent garment 5 and the inguinal tissues 185. Similarly, the rear straps 20 (see FIG. 5) can have a cinch 355 to allow tensile forces within the rear straps 20 to be adjusted and can have the rear straps 20 extending through a strap channel 380 and can apply a rear strap normal force 163 (see FIG. 5) from the belt 25 to the separator pad 30 against the gluteal tissues (see FIG. 5) to place the absorbent undergarment into a fluid-impermeable contact 162 or fluid-impermeable seal 162 with the gluteal tissues to prevent escape of exudate material across the fluid-impermeable contact 162 as described in the specification for FIG. 5.

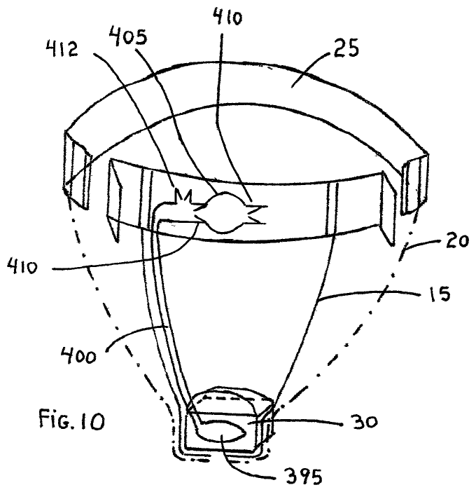

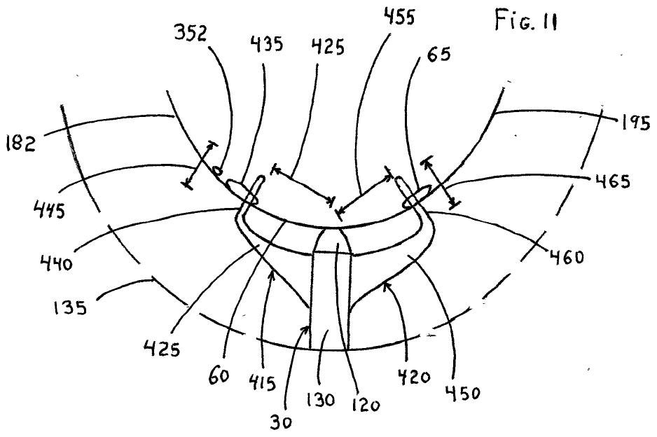

Various materials have been contemplated for the separator pad 30 including polymeric foam (closed cell and open cell), natural fibrous materials, polymeric fibrous materials, and other composite materials. Additionally, an inflatable balloon 395 can be used as the separator pad 30 or as a portion of a separator pad 30. As shown in FIG. 10, for example, an inflatable balloon 395 could be placed within the interior or adjacent to the foam separator pad 30 that has been described in this specification. The inflatable bulb 405 can be connected via a small diameter tube 400 to an inflation bulb 405 located on the belt 25. The inflation bulb 405 can consist of two one-way inflation valves 410 located at each end of the inflation bulb 405. To provide a greater force of the separator pad 30 against the perineal tissues 60 the individual could compress the squeeze bulb 405 to inflate the balloon 395 further thereby increasing the compressive force 115 of the balloon 395 and the separator pad 30 against the perineal tissue 60.

Pressure within the balloon 395 can be decreased by squeezing a one-way deflation valve 412 thereby decreasing the compressive force 115 that the balloon 395 and separator pad 30 place onto the perineal tissues 60. The balloon can be formed from a polymeric material including Pebax, polyurethane, silicone, latex, polyethylene, or other elastomeric or semi-elastic polymer used in medical device having a balloon, for example. An inflatable balloon 395 can alternately be coated with a soft or porous polymeric material and used as a separator pad 30 that makes direct contact with the perineal tissues; the jacket 135 and straps 15, 20 can be bonded to the balloon via intermediate connecting members comprised of soft polymeric foam, for example, that are placed between the balloon 395 and the jacket 135 or straps 15, 26.

As shown in FIG. 11, a portion of the absorbent garment can have a front positioning member 415 (FPM) or a rear positioning member 420 (RPM) or both a FPM 415 and an RPM 420 attached to the separator pad 30 as shown, or the positioning members (FPM 415 or RPM 420) can be attached to the jacket 135 or other structures adjacent to the support pad. The front positioning member 415 (FPM) can have a FPM arm 425 that attaches, for example, to the pad base via an adhesive bond, solvent bond, thermal bond or can be contiguous with the pad base. The FPM arm 425 can extend with an FPM arm length 430 of 1-1.5 cm (range 0.5-5 cm) in a direction from the separator pad 30 toward the vaginal opening 435. The FPM 415 can have an FPM probe 440 attached, bonded, or contiguous with the FPM arm 425 that extends from the FPM arm 425 towards and into the vaginal opening 435 with an FPM probe length 445 of 1-2 cm (range (0.5-3 cm). The FPM arm 425 and FPM probe 440 are formed from flexible materials including polymeric materials, foam materials, fibrous materials or other materials that can contact the skin without causing irritation. Materials used in the construction of women's tampons can be used, for example, to form the FPM probe 440. Placing the FPM probe 440 into the vaginal opening 435 can assist in positioning the separator pad 30 in a location between the anus 65 and the genitalia 70 thereby separating fecal material from access to the urethra and preventing urinary tract infection, for example.

The rear positioning member 420 (RPM) can have an RPM arm 450 that attaches, for example, to the pad base via an adhesive bond, solvent bond, thermal bond or can be contiguous with the pad base. The RPM arm 450 can extend with an RPM arm length 455 of 1-1.5 cm (range 0.5-5 cm) in a direction from the separator pad 30 toward the anus 65. The RPM 420 can have an RPM probe 460 attached, bonded, or contiguous with the RPM arm 450 that extends from the RPM arm 450 towards and into the anus 65 with a RPM probe length 465 of 1-2 cm (range (0.5-3 cm). The RPM arm 450 and RPM probe 460 are formed from flexible materials including polymeric materials, foam materials, fibrous materials or other materials that can contact the skin without causing irritation. The RPM probe 460 can have a flexibility that allows the RPM probe 460 to be pushed out of the anus 65 via a bowel movement. Materials used in the construction of the RPM probe 460 include polyurethane, silicone, or other flexible and soft polymeric materials including foam, fibers or natural fiber materials (i.e., found in nature, for example). Placing the RPM probe 460 into the anus 65 can assist in positioning the separator pad 30 in a location between the anus 65 and the genitalia 70 thereby separating fecal material from access to the urethra and preventing urinary tract infection, for example.

Reference names and structural descriptions used in various embodiments of the present invention and found within this patent specification can be applied equally with other embodiments of the present invention. Other embodiments of the present invention are contemplated and the embodiments presented are not intended to limit the scope of the present invention. Reference names that are common to multiple embodiments are understood to have the same meaning or interpretation.

1. An absorbent undergarment for absorbing and collecting fecal exudate from an anus and urine from a urethra of an individual, said absorbent undergarment comprising;
A. a separator pad having a tissue contact surface positioned into contact with perineal tissues between the anus and genitalia of the individual, said separator pad formed from a material that undergoes a deformational strain upon application of a compressive force of said separator pad against the perineal tissues,
B. a pad support comprised of four elastic straps and a belt, said belt extending around a waist of the individual, said four elastic straps being attached to said belt and to said separator pad, said four elastic straps comprised of two front straps and two rear straps, each of said two front straps extending along and applying a front strap normal force against inguinal tissues of the individual from said belt to said separator pad, each of said two rear straps extending along and applying a rear strap normal force against gluteal tissues of the individual from said belt to said separator pad,
C. said four elastic straps providing tensile forces onto said separator pad to compress said separator pad with said compressive force to form a fluid-tight seal of said separator pad with the perineal tissues,
D. a jacket comprised of a fluid-impermeable cover on a garment side of said absorbent undergarment, a fluid-permeable liner on a body side of said absorbent undergarment, and a fluid-absorbent layer located between said cover and said liner, said jacket being located between and attached to each of said two front straps and extending from said belt along the inguinal tissues to said separator pad, said jacket further being located between and attached to each of said two rear straps extending along the gluteal tissues from said belt to said separator pad, said front straps and said rear straps forming a fluid-impermeable contact of said absorbent undergarment with the inguinal and gluteal tissues,
E. whereby the fecal exudate is unable to pass across said separator pad into contact with the urethra and is unable to pass across said fluid-impermeable contact of said absorbent undergarment with the inguinal and gluteal tissues.

2. The absorbent undergarment of claim 1 wherein said front straps provide a forward component and lateral component of said tensile forces to said separator pad, and rear straps provide a rearward component and lateral component of said tensile forces to said separator pad to maintain position of said separator pad between the anus and the genitalia.

3. The absorbent undergarment of claim 1 wherein said separator pad is formed from a material configured to undergo said deformational strain of at least 10% when exposed to said compressive force of two pounds or less to thereby generate said leak-tight seal of said separator pad with the perineal tissues.

4. The absorbent undergarment of claim 1 wherein said four elastic straps are contained within strap channels extending from said belt to said separator pad.

5. The absorbent undergarment of claim 1 wherein said four elastic straps are contained within channels located within said jacket.

6. The absorbent undergarment of claim 1 wherein said belt is attached to two or more of said four elastic straps via cinches, said cinches being configured to hold said tensile forces of each of said two or more of said four elastic straps and release said tensile forces via activation of said cinches by the individual, said cinches allowing the individual to adjust said tensile forces in said two or more of said four elastic straps, thereby altering said compressive forces of said separator pad onto the perineal tissue and provide for positioning of said separator pad between the anus and the genitalia.

7. The absorbent undergarment of claim 1 further comprising a garment rim, said garment rim being attached to said jacket and extending laterally outwards from said attachments of said jacket to said four elastic straps, said garment rim further extending around each leg of the individual, said garment rim having an elastic band that makes contact with the leg, said elastic band of said garment rim and said fluid-impermeable contact of said absorbent undergarment with the inguinal and gluteal tissues providing two modes of preventing exudate from exiting said absorbent undergarment.

8. The absorbent undergarment of claim 1 further comprising a handle located on the garment side of said undergarment adjacent to said separator pad, said handle allowing the individual to position said separator pad relative to the perineal tissue.

9. The absorbent undergarment of claim 1 wherein said separator pad is formed in part from a compressible foam.

10. The absorbent undergarment of claim 9 where said compressible foam comprises a closed cell foam.

11. The absorbent undergarment of claim 1 wherein said separator pad comprises an inflatable balloon, said inflatable balloon being connected by a tube to an inflation bulb attached to said belt, said bulb being configured to be squeezed by the individual to inflate said balloon, said balloon being inflatable to cause said separation pad to alter said compressive force against the perineal tissues.

12. The absorbent undergarment of claim 1 wherein said separator pad has a pad tip and a pad base, said pad tip providing said deformational strain of said separator tip against said perineal tissue; said pad base providing a surface for attachment of said four straps to said separator pad and providing a barrier to prevent fecal exudate from access to the urethra.

13. The absorbent undergarment of claim 1 wherein said belt is a tapered belt having a shorter upper length than a lower length, said belt configured to be positioned above a hip of an individual and prevent downward movement.

14. The absorbent undergarment of claim 1 wherein said belt has a tacky coating applied to the body side of said belt, said tacky coating taken from a group comprising silicone, polyurethane, and latex.

15. The absorbent undergarment of claim 1 further comprising a front positioning member, said front positioning member being permanently attached or contiguous with said separator pad, said front positioning member comprising a front probe configured to extend within a vaginal opening, said front positioning member configured to prevent movement of said separator pad in a forward or rearward direction.

16. The absorbent undergarment of claim 1 further comprising a rear positioning member, said rear positioning member permanently attached or contiguous with said separator pad, said rear positioning member comprising a rear probe configured to extend within an anus, said rear positioning member configured to prevent movement of said separator pad in a forward or rearward direction.

17. The absorbent undergarment of claim 1 wherein said separator pad has a separator pad height of one inch ranging from ½ inch to 2 inches.

18. The absorbent undergarment of claim 1 wherein said belt has belt openings on each side of said belt forming a belt front portion and a belt rear portion, said front straps being attached to said belt front portion and said rear straps being attached to said belt rear portion.

19. An absorbent undergarment for absorbing and collecting fecal exudate from an anus of an individual into a rear compartment and urine from a urethra of an individual into a front compartment, said absorbent undergarment comprising;
A. a separator pad comprised of a separator pad tip and a separator pad base, said separator pad tip having a tip contact surface that makes a fluid-tight seal with perineal tissues, said separator pad tip being positioned between the anus and genitalia of the individual, said separator base being located adjacent to said separator pad tip downward from the perineal tissues,
B. said separator pad tip formed from a material that undergoes a deformation upon application of a compressive force applied by the separator pad against the perineal tissues, said separator pad material having a resiliency to return to an original configuration upon release of said compressive force,
C. a pad support comprised of four elastic straps and a belt, said belt extending around a waist of the individual, said four straps being attached to said belt and attached to said separator pad base, said four elastic straps comprised of two front straps and two rear straps, each of said two front straps extending along the inguinal region of the individual from said belt to said separator pad, each of said two front straps exerting a normal force against the inguinal region, each of said two rear straps extending rearward along gluteal tissues of the individual from said belt to said separator pad, each of said two rear straps exerting a normal force against the gluteal tissues, said front straps providing forward and lateral tensile forces to said separator pad, and said rear straps providing rearward and lateral tensile forces to said separator pad to maintain position of said separator pad between the anus and the genitalia,
D. said four elastic straps providing said forward, rearward, and lateral tensile forces onto said separator pad tip to compress said separator pad tip with said compressive force to form said fluid-tight seal of said separator pad tip with the perineal tissue,
E. a jacket comprised of a cover impermeable to fluid passage therethrough on the garment side of said jacket, a liner that is permeable to fluid passage therethrough on the body side of said jacket, and a fluid-absorbent layer able to absorb and hold fluid located between said cover and said liner, said jacket forming a part of said front compartment being located between and attached to said front straps extending from said belt to said separator pad, said jacket further forming a part of said rear compartment being located between and attached to said rear straps extending from said belt to said separator pad, said absorbent undergarment making a fluid-impermeable contact with the inguinal tissues and gluteal tissues from said belt to said separation pad,
F. said normal forces of said front straps and said rear straps providing said absorbent garment with a fluid-impermeable contact of said absorbent garment with the inguinal and gluteal tissues,
G. whereby the fecal exudate is unable to pass from said rear compartment to said front compartment and the fecal exudate and urine are unable to pass between said absorbent undergarment and the inguinal tissues or gluteal tissues.

20. An absorbent undergarment for absorbing and collecting fecal exudate from an anus and urine from a urethra of an individual, said absorbent undergarment comprising;
A. a separator pad having a contact surface positioned against perineal tissue between the anus and genitalia of the individual, said separator pad formed from a material that undergoes a deformational strain upon application of a compressive force by said separator pad against the perineal tissues,
B. a pad support comprised of elastic straps and a belt, said belt extending around a waist of the individual, said elastic straps being attached to said belt, said elastic straps comprised of at least two front straps and at least two rear straps, said at least two front straps each extending along and applying a force against an inside aspect of a leg of the individual from said belt to said separator pad, said at least two rear straps each extending along and applying a force against an outside aspect of a leg from said belt to said separator pad,
C. said elastic straps providing tensile forces onto said separator pad to compress said separator pad with said compressive force to form a fluid-tight seal of said separator pad with the perineal tissue,
D. a jacket comprised of a fluid-impermeable cover on the garment side of the jacket, and a fluid-absorbent layer located on the body side of said fluid-impermeable cover, said jacket being attached to said front straps and extending from said belt to said separator pad, said jacket further being attached to said rear straps extending from said belt to said separator pad,
E. whereby the fecal exudate is unable to pass across said separator pad into contact with the urethra.
